(19) United States
(12) Patent Application Publication
(10) Pub. No.: US 2006/0270974 A1
(43) Pub. Date: Nov. 30, 2006

(54) Methods and systems for filtering aspirated materials

(75) Inventors: Thomas G. Goff, Menlo Park, CA (US); Mark C. Yang, San Francisco, CA (US); Michael J. Orth, Morgan Hill, CA (US); Gary Hulme, San Jose, CA (US); John M. MacMahon, Mountain View, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: KERBEROS PROXIMAL SOLUTIONS, INC., Cupertino, CA (US)

(21) Appl. No.: 11/131,011

(22) Filed: May 16, 2005

(51) Int. Cl. A61M 31/00; A61M 37/00

(52) U.S. Cl. 604/93.01; 604/506

(57) ABSTRACT

Solid materials are separated from hollow body structure aspirates using a filter assembly disposed between an aspiration catheter and an aspirate receptacle. Filter elements having different pore or mesh sizes may be used to size classify the separated solid materials. Multiple filter assemblies may be disposed in parallel or series between the aspiration catheter and aspirate receptacle to provide for different levels of size classification.

### BACKGROUND OF THE INVENTION

1. Field of the Invention

The present invention relates generally to medical apparatus and methods. More particularly, the present invention relates to a method and system for separating and optionally classifying solids removed from a patient in a fluid aspirate.

Aspiration is a part of many surgical procedures performed in various body structures and lumens. Blood and/or other natural body fluids may be aspirated from various hollow body structures, such as blood vessels, cysts, pseudocysts, abscesses, blood vessel grafts, lung passages, bile ducts, ureters, urethras, fallopian tubes, ear canals, the gastrointestinal tract, and the like. In some instances, aspiration is performed on a natural body fluid(s) only, while in many other instances, a liquid irrigant will be introduced which will form at least part of the aspirated fluid. Such irrigants may comprise saline or other biologically inert fluids. Alternatively, such irrigants may comprise biologically active agents, such as thrombolytic agents introduced to occluded blood vessels, antiseptic or antibiotic agents introduced to infected body locations, or the like.

Of particular interest to the present invention, fluids aspirated from any of these hollow body structures will often contain solid materials, such as cellular debris, damaged tissue, thrombus, or the like, which is aspirated together with the fluid. In many instances, such removed solid materials will have diagnostic or other value to a treating physician. For example, during aspiration, it may be desirable to monitor the solid materials which are being removed in order to decide when to terminate or alter or adjust the aspiration protocol. Additionally, the identification of the aspirated material may serve as a diagnostic tool to direct further intervention or other therapies.

While the collected materials may be observed in the aspirate collection bags which are commonly employed in such procedures, it will usually be difficult to remove the materials while additional aspirate is entering the collection bags and the solids will frequently remain suspended and difficult to separate from the collected materials.

For these reasons, it would be desirable to provide improved and additional systems and protocols for separating solid materials from liquid and fluid aspirates removed from a hollow body structure. It would be particularly desirable if the methods and protocols permitted direct observation and/or removal of the separated solid materials while an aspiration protocol was continuing. It would be further desirable if the separated solid materials were collected in a form substantially separated from a liquid fraction of the materials removed from the hollow body structure and further that the separated solid materials be in a convenient structure or assembly to permit easy removal and observation. In some instances, it would also be desirable to provide for classification of the solid materials, i.e. separation based on size, while the aspiration protocol was being performed. At least some of these objectives will be met by the inventions described and claimed hereinbelow.

2. Description of the Background Art

Patient irrigation and aspiration systems which may employ the separation technology of the present application are described in commonly assigned U.S. Pat. Nos. 6,827,701 and 6,878,128, the full disclosures of which are incorporated herein by reference.

### BRIEF SUMMARY OF THE INVENTION

In a first aspect of the present invention, methods are provided for separating materials in fluid aspirates removed from a hollow body structure. The fluid is aspirated from the hollow body structure, where the fluid carries entrained solid materials in the aspirate. The solid materials are filtered from the fluid to produce both a filtrate and a fluid stream. The fluid stream is collected separately from the filtrate. In this way, the filtrate may be easily observed and optionally removed from the aspiration circuit even while the aspiration protocol continues.

The fluid may be aspirated from a variety of hollow body organs and other structures, including blood vessels, cysts, pseudocysts, abscesses, blood vessel grafts, lung passages, bile ducts, ureters, urethras, fallopian tubes, ear canals, joint capsules, the gastrointestinal tract, and the like. Thus, natural body fluids which may be aspirated according to the present invention include blood, bile, urine, synovial fluid, and the like. In addition to such natural body fluids, the hollow body structures may optionally be irrigated prior to and/or during aspiration. The introduction of irrigation fluid may improve debris capture through the aspiration channel by creating localized mixing/turbulence and possibly decreasing the viscosity of the aspirant. Thus, the aspirated fluids may comprise or consist of a variety of irrigant fluids introduced to the hollow body structure. Suitable irrigant fluids include saline, lactated ringers, and the like. The irrigant fluids may further comprise active agents intended for therapeutic or diagnostic purposes. For example, in the case of occluded blood vessels, thrombolytic agents may be introduced as part of an irrigant stream. Alternatively, in the case of infected hollow body structures, the irrigant may include antibiotics, antiseptics, or the like.

Most typically, aspiration will be performed by introducing an aspiration catheter, cannula, or other tubular or needle-like device into the hollow body structure. By applying a vacuum to a proximal end of the aspiration device, the fluid may be withdrawn through a port or ports at or near the distal end of the device which has been placed within an interior region of the hollow body structure. For convenience, as used hereinafter and in the claims, the aspiration structure will be referred to as a “catheter,” but it will be appreciated that this term is intended to be broad enough to encompass needles, cannulas, tubular structures, conduits, and other aspiration structures known in the medical art.

The aspirated fluids will usually be collected in an aspirate receptacle, such as a conventional fluid collection bag. A syringe, vacuum connection, or other conventional vacuum source may be connected at or through the catheter and/or the aspiration receptacle in order to aspirate the fluid from the hollow body structure, through the aspiration catheter, and into the aspirate receptacle. The filter(s) may be positioned before or after the aspiration source.

In a preferred aspect of the present invention, at least one filter assembly including a filter housing and a removable (and replaceable) filter element is placed between the aspiration catheter and the aspirate receptacle in order to remove solid materials from the aspirate before the remaining liquid phase of the aspirate flows to the aspirate receptacle. The filter element may be any conventional filter element, such as a paper, polymer, a woven filter membrane, a screen, other porous member, or the like. The filter element may have any one of a variety of geometries including cup-shaped, conical and the filter could be inclined or slanted in the filter-housing to spread the filtrate over the filter to permit differentiation of the filtrate material. Additionally, the filter element could be coated or otherwise combined with a chemical, biological, or other indication or marker to facilitate identification of different analytes or markers present in the filtrate, typically using colormetric indicating systems. Preferably, the filter element will allow the separated solid materials to collect on an exposed surface so that the solid materials will be easily removed and/or absorbed. The filter element will have a pore size or screen size selected to separate solid particles at a desired particle size cutoff. For example, suitable filter membranes may have a pore size in the range from 1 μm to 1000 μm, usually from 5 μm to 240 μm, and preferably from 20 μm to 120 μm. For separation of larger particles, screens having mesh sizes in the range from 0.1 mm to 5 mm, and preferably from 0.2 mm to 1 mm may be used.

In a specific aspect of the methods of the present invention, at least two filter assemblies may be provided between the aspiration catheter and the aspirate receptacle. The two or more filter assemblies may be disposed in parallel, in series, or in a combination of parallel and series arrangements. Typically, valving will be provided so that the multiple filter assemblies may be isolated from the flowing stream of aspirate so that the filter elements may be removed and the collected solids observed even while the aspiration protocol continues. In some cases, an unfiltered bypass path will be provided with valving so that a single filter assembly or group of filter assemblies may be isolated while the aspirate is directed or shunted to the aspirate receptacle without any filtration.

In a further specific aspect of the methods of the present invention, two or more filter elements may be employed in series or in parallel in order to size classify the materials being removed. Most simply, two, three, or more filter elements having progressively smaller pore or mesh sizes may be provided in series in a single filter housing. Alternatively, such a series of progressively smaller filtering elements could be provided in separate filter assemblies which are disposed in series between the aspiration catheter and the aspirate receptacle. Alternatively, separate filter assemblies having filter elements with differing particle size cutoffs could be provided in parallel or in a series-parallel arrangement in order to separately collect and classify particles having different sizes. In any of these ways, the solid materials may be filtered and separated into at least two size groups, often at least three size groups, and into virtually any number of different sized collection groups desired.

In a second aspect of the present invention, systems for aspirating a hollow body structure comprise an aspiration catheter, an aspirate receptacle connectable to receive aspirate from the aspiration catheter, and at least one filter assembly disposed between the aspiration catheter and the aspirate receptacle. Typically, the aspirate receptacle will be a fluid collection bag, although any other conventional medical receptacle would be suitable. The aspirate receptacle will typically be connected by a flexible tube between the aspiration catheter and the aspirate receptacle. Usually, a syringe, vacuum connector, or other conventional vacuum source will be provided in order to effect the system aspiration.

The filter assembly usually comprises at least one filter housing having at least one filter element removably disposed in an interior thereof. The filter element, as described above, may comprise a filter membrane, a mesh, link, or the like, having a pore or mesh size selected to collect and separate solids having a target threshold size or sizes. The filter housing and optionally filter element may be at least partially transparent to permit observation of the solid materials as they collect. In an exemplary embodiment, the filter housing has an upper shell and a lower shell which may be taken apart to permit introduction, removal, and replacement of the filter element in the interior of the housing. The housing will further have conventional connectors to permit connection at an upper end to the aspiration catheter and at a lower end to the aspirate receptacle.

The system may further comprise at least a second filter assembly, a third filter assembly, or even greater number of filter assemblies which may be disposed in parallel or series to the first filter assembly. Additionally, at least one unfiltered flow path may be provided in parallel to the filter assembly(ies), and valving will be provided to permit selective flow through any one or more of the filter assemblies as well as through the unfiltered flow path. The different filter assemblies may each have a filter element with a different pore or mesh size. Alternatively, two or more filter elements may be provided within a single filter assembly, where the individual filter elements within the individual assembly may optionally have different pore or mesh sizes.

### BRIEF DESCRIPTION OF THE DRAWINGS

FIG. 1 illustrates an exemplary system including an irrigant source, an aspiration and irrigation catheter, a filter assembly, an aspirate receptacle, and a vacuum source, constructed in accordance with the principles of the present invention.

FIG. 2 illustrates an exemplary filter housing constructed in accordance with the principles of the present invention.

FIGS. 3 and 4 illustrate two exemplary filter assembly connection patterns which may be employed in the apparatus and methods of the present invention.

FIG. 5 illustrates use of filter elements having different pore or mesh sizes disposed in series for classifying solid materials in accordance with the principles of the present invention.

FIG. 6 illustrates the use of filter elements having different pore or mesh sizes disposed in parallel for classifying solid material sizes in accordance with the principles of the present invention.

### DETAILED DESCRIPTION OF THE INVENTION

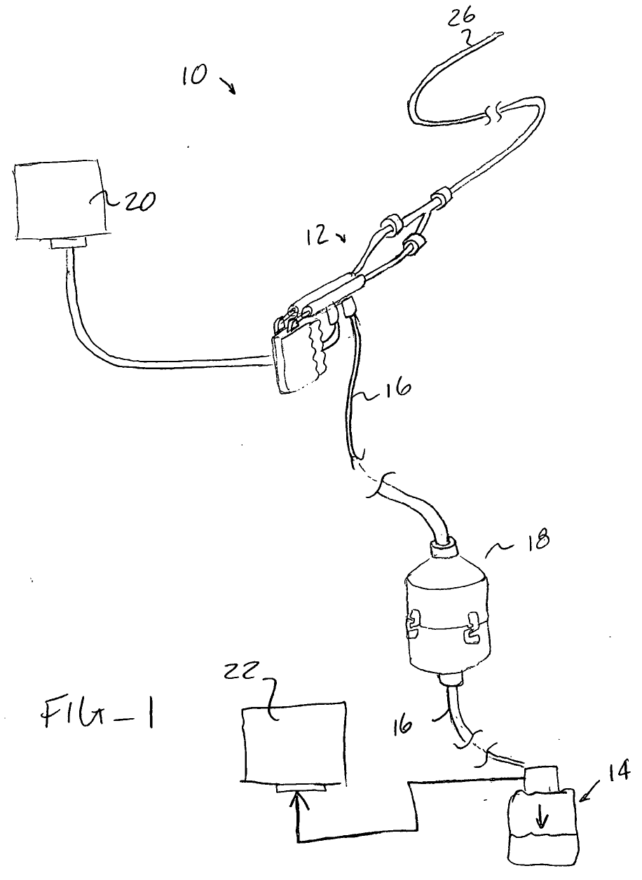

The present invention provides systems and methods for separating solid materials, typically particulate materials, from aspirated body fluids, including both natural body fluids and introduced fluids. As shown in FIG. 1, an exemplary system 10 comprises catheter 12 (move the arrow for 12 distal to the 2-line section) which is connected to an aspirate receptacle 14 by tubing 16. The catheter 12 is illustrated as an irrigation/aspiration catheter which is connectable to a source of irrigant fluid 20. Exemplary irrigation/aspiration catheters are described in commonly assigned U.S. Pat. Nos. 6,827,701 and 6,878,128, both of which have been previously incorporated herein by reference. It will be appreciated, however, that the present invention does not require that the catheters 12 provide for irrigation, but rather only that they provide for aspiration and the ability to discharge a fluid aspirate stream to an aspirate collection receptacle.

The irrigation/aspiration catheter 12 illustrated in FIG. 1 and described in the copending U.S. patents incorporated above, provides for a pair of syringe elements for both introducing the irrigant fluid from the irrigant source 20 and discharging the aspirate stream to the aspirate receptacle 14. In other cases and for other aspiration catheters, it may be desirable to provide a separate vacuum source 22 which may be connected to or through the catheter 12 and/or aspirate receptacle 14 in order to draw the aspirate stream through the tubing 16 or other discharge connections. The present invention, of course, does not depend on what particular mechanism is provided for generating the aspirate stream or the location of the said mechanism with respect to the filter(s).

The catheter 12 has a distal end or portion 26 which is introducible into a target hollow body structure in order to withdraw fluid therefrom to produce the aspirate stream. In the preferred example of the irrigation aspiration catheter, described in the previously incorporated commonly owned U.S. patents, the catheter 12 will be intended for introduction to a blood vessel for introducing a thrombolytic agent in order to disrupt clot. In this exemplary use, the solid material aspirated by the catheter will frequently comprise disrupted clot, thrombus, and/or plaque, which is then discharged through the aspirate line 16 to the aspirate receptacle. It will be appreciated, however, that the present invention is not limited to vascular use, clot disruption, or any other particular treatment protocol, and may instead extend to the different hollow body structures and body fluids described above.

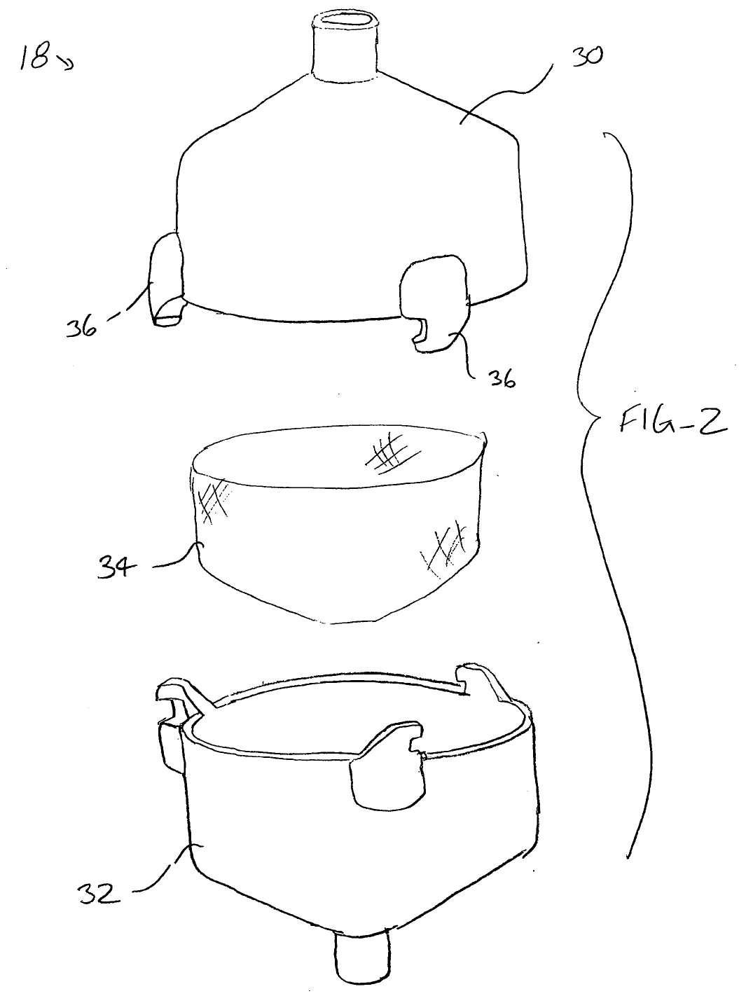

In the simplest embodiment of the present invention, a single filter housing 18 is disposed between the aspiration catheter 12 and the aspirate receptacle 14, as illustrated in FIG. 1. As shown in FIG. 2, the filter assembly 18 typically comprises an upper shell 30, a lower shell 32, and a filter element 34 which may be disposed within the interior of the shells. The upper shell 30 is removable from the lower shell 32, typically including mating connectors 36 disposed about the open peripheries of each shell. All or a portion of the shells 30 and 32, as well as optionally the filter element 34, may be composed of transparent materials in order to permit observation of the collection of solids within the filter element 34 as the aspiration progresses. While the particular structure of filter assembly 18 shown in FIG. 2) is suitable and presently preferred, a variety of other specific filter assembly constructions could also be used.

The filter elements 34 may comprise any one of a variety of conventional filtering materials, as generally described above in the Summary of the Invention. The geometries in which the filter elements 34 are arranged will depend in large part on the construction of the remainder of the filter assembly. In the embodiment of FIG. 18, the filter element 34 is constructed so that it nests within the lower shell 32 of the filter assembly. A wide variety of other geometries would also be suitable.

In many instances, it will be desirable to provide two or more filter assemblies 18 between the aspiration catheter 12 and the aspirate receptacle 14. As illustrated in FIG. 3, a pair of filter assemblies 18 are disposed in parallel to receive the aspirate through an upper portion of tubing 16 and discharge the aspirate through a lower portion of tubing 16. Isolation valves 40 are provided so that either of the filter assemblies 18 may be taken off-line to permit access even while the aspiration continues. Optionally, as shown in broken line, a third filter assembly 18 may also be provided in parallel. It is clear that any number of such filter assemblies may be provided in parallel, although the valving may have to be modified in order to permit any single one of the assemblies to be isolated while all others remain on line.

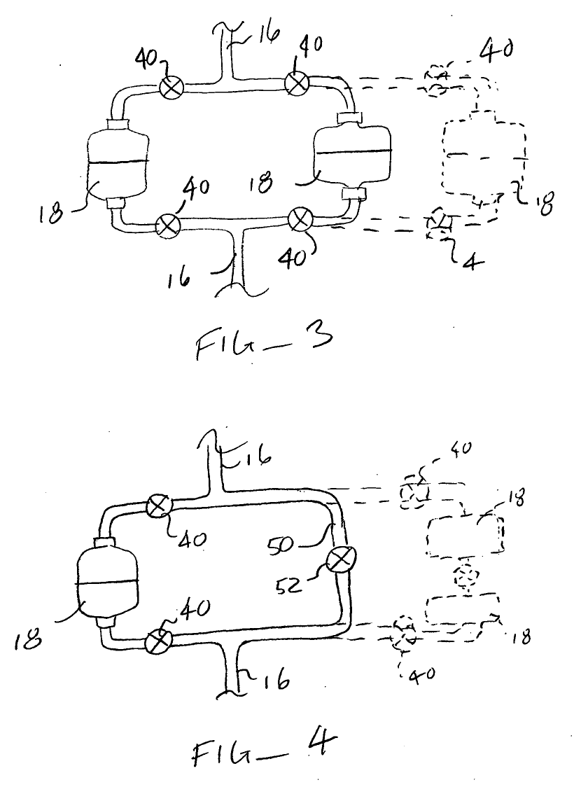

The filter assembly arrangement of FIG. 4 illustrates another arrangement within the scope of the present invention. A first filter assembly 18 may be provided and isolated by valves 40 in parallel with an unfiltered flow path 50 having a valve 52 therein. With this embodiment, all flow could be directed through the filter assembly 18 with valve 52 being closed. Should it be desired to gain access to filter 18, the isolation valves 40 could be closed and the flow path valve 52 open.

As a still further option, as shown in broken line in FIG. 4, additional filter assemblies 18 could be provided, with individual assemblies 18 being disposed in series, where the two series assemblies may be together placed in parallel with first filter assembly 18. It will be appreciated that a wide variety of different parallel and/or series arrangements of filter assemblies and unfiltered flow paths may be provided within the scope of the present invention.

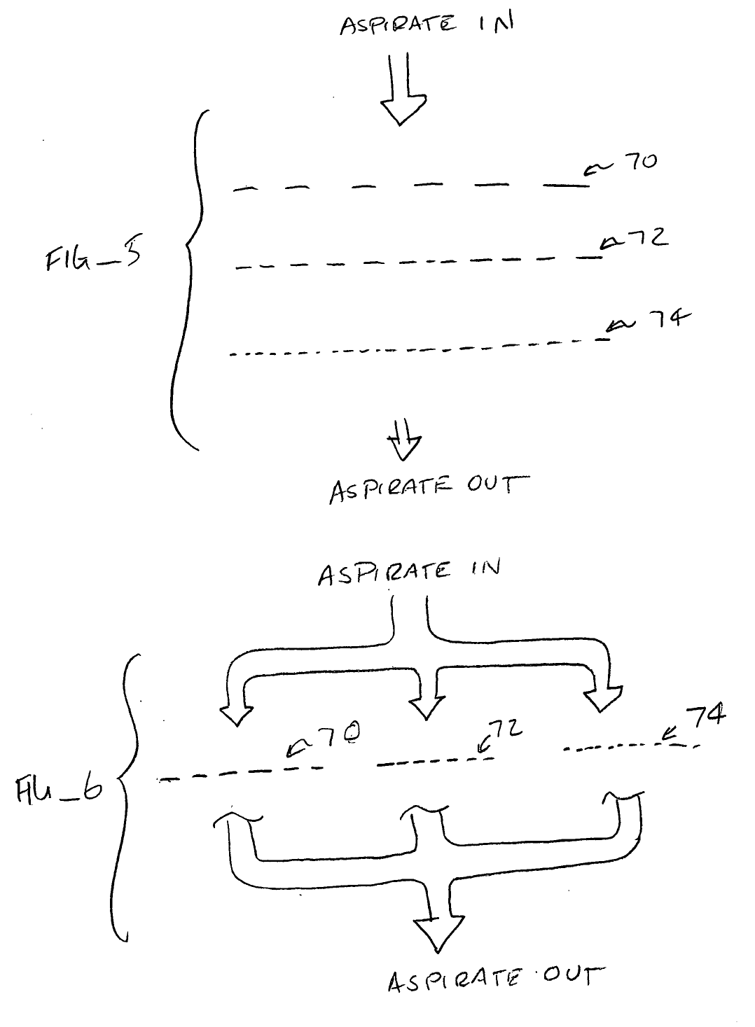

Referring now to FIGS. 5 and 6, it should also be appreciated that filter elements having different pore or mesh sizes may also be provided in series and/or in parallel in order to permit separation and classification of the particulate solid materials which are being separated from the flowing aspirate. For example, as shown in FIG. 5, three filter elements 70, 72, and 74 may be placed in series, with progressively smaller pore or mesh sizes in the direction of flow. Thus, all particles having a size greater than a first threshold would collect on top of the first filter element 70, while intermediate particle sizes having a smaller threshold size would collect on the second filter element 72. Still smaller particles would collect on the third filter element having the smallest pore or mesh sizes, while still smaller particles would pass into the aspirate receptacle without separation. The three filter elements 70, 72, and 74 could be arranged within a single filter housing or alternately within a series of three filter housings.

The filter elements 70, 72, and 74 could also be arranged in parallel, as shown in FIG. 6. Each of the screens would collect particles having a size greater than the threshold pore or mesh size of the filter element. Such a parallel arrangement of the different sized filter elements, however, would not result in true size classification, since each of the filters would collect the larger elements and would only allow smaller elements to selectively pass. This configuration would allow the use of indicator media which would be placed in the filter(s). This indicator(s) could detect the presence of a certain biological or chemical components in the aspirant or filtered solids for the diagnostic purposes.

The collected elements could be used for a variety of therapeutic purposes. For example, when collecting aspirated thrombus in thrombolytic procedures,

The collected material could be used to diagnose certain disease states or conditions. These diagnostic findings could then be used to direct further interventions and/or treatments.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A method for separating materials removed from a hollow body structure, said method comprising:
aspirating a fluid from the hollow body structure, wherein solid materials from the hollow body structure are entrained in the aspirate;
filtering solids from the aspirate to produce a filtrate and a fluid stream; and
collecting the fluid stream separately from the filtrate.

2. A method as in claim 1, wherein aspirating the fluid comprises introducing an aspiration catheter in the hollow body structure.

3. A method as in claim 1, wherein filtering comprises interposing at least one filter assembly including a filter housing and a removable filter element between the aspiration catheter and an aspirate receptacle.

4. A method as in claim 3, further comprising removing the filter element from the housing to permit inspection of solids collect by the filter element.

5. A method as in claim 4, wherein the filter element is removed while aspirate continues to flow from the aspiration catheter to the aspirate receptacle.

6. A method as in claim 5, wherein the aspirate flow is directed past the filter assembly while the filter element is removed.

7. A method as in claim 6, wherein the aspirate is directed past the filter assembly through at least a second filter assembly disposed in parallel to the first filter assembly.

8. A method as in claim 6, wherein the aspirate is directed past the filter assembly through an unfiltered flow path disposed in parallel to the filter assembly.

9. A method as in claim 1, wherein filtering comprises separating the solids into at least two size groups.

10. A method as in claim 1, wherein filtering comprises separating the solids into at least three size groups.

11. A method as in claim 1, wherein filtering comprises passing the fluid through at least two filter elements, wherein said filter elements have different pore sizes.

12. A method as in claim 11, wherein the at least two filter elements are arranged in series.

13. A method as in claim 11, wherein the at least two filter elements are arranged in parallel.

14. A method as in claim 1, wherein the hollow body structure is selected from the group consisting of blood vessels, cysts, pseudocysts, abscesses, blood vessel grafts, lung passages, bile ducts, ureters, urethras, fallopian tubes, ear canals, and gastrointestinal tracts.

15. A method as in claim 14, wherein the hollow body structure is an artery.

16. A method as in claim 15, wherein the artery is a coronary artery, a peripheral artery, or a cerebral artery.

17. A method as in claim 14, wherein the hollow body structure is a vein.

18. A method as in claim 17, wherein the vein is a peripheral vein.

19. A method as in claim 1, further comprising introducing an irrigation fluid to the hollow body structure, wherein at least a portion of the aspirated fluid comprises the irrigation fluid.

20. A method as in claim 19, wherein aspirating and introducing the irrigation fluid are performed with an irrigation and aspiration catheter positioned in the hollow body structure.

21. A method as in claim 19, wherein the irrigation fluid comprises saline.

22. A method as in claim 1, wherein the irrigation fluid comprises a biologically active agent.

23. A method as in claim 22, wherein the biologically active agent comprises a thrombolytic agent.

24. A system for aspirating a hollow body structure, said system comprising:
an aspiration catheter;
an aspirate receptacle connectable to receive aspirate from the aspiration catheter;
at least one filter assembly disposed between the aspiration catheter and the aspirate receptacle.

25. A system as in claim 24, wherein the aspirate receptacle is a fluid collection bag.

26. A system as in claim 24, further comprising a flexible tube connecting the aspiration catheter to the aspirate receptacle, wherein the filter housing is connected to the flexible tube between the catheter and the receptacle.

27. A system as in claim 24, wherein the filter assembly comprises:
at least one filter housing; and
at least one filter element removably disposed in the filter housing to separate solids from aspirate flowing from the irrigation and aspiration catheter to the aspirate receptacle.

28. A system as in claim 26, wherein the filter housing is at least partly transparent to allow observation of the filter contents.

29. A system as in claim 26, wherein the filter housing comprises an upper shell and a lower shell, wherein the upper shell and lower shell are separable to provide access to the filter element in an interior of the filter housing.

30. A system as in claim 27, further comprising at least a second filter assembly disposed in parallel and/or series to the first filter assembly between the irrigation and aspiration catheter and the aspirate receptacle, and valving to permit selective diversion of aspirate flow through either filter assembly.

31. A system as in claim 30, wherein the second filter assembly has a filter element with a pore size different from that of the first filter assembly.

32. A system as in claim 27, wherein the filter assembly includes at least two filter elements, wherein at least one of said filter elements has a different pore size than that of another filter element.

33. A system as in claim 27, further comprising at least one flow path in parallel to the filter assembly, and valving to permit selective bypass of the filter assembly.

34. A system as in claim 24, wherein the aspiration catheter comprises a catheter shaft having at least one irrigation lumen, at least one aspiration lumen, at least one irrigation port near a distal end of the shaft, and at least one aspiration port near said distal end.

35. A system as in claim 34, wherein the distal end of the shaft is free from isolation balloons.

36. A system as in claim 35, wherein the distal end of the shaft includes at least one isolation balloon disposed proximally of the irrigation and aspiration ports.

37. A system as in claim 34, wherein the distal end of the shaft includes at least a second isolation balloon disposed proximally of the irrigation and aspiration ports.
